(19) United States
(12) Patent Application Publication
(10) Pub. No.: US 2013/0177627 A1
(43) Pub. Date: Jul. 11, 2013

(54) Growth inhibitory effects of nanoparticles containing triterpene glycosides or triterpenes

(71) Applicants: Linda Einbond, Crestwood, NY (US); Stepehn Redenti, New York, NY (US)

(72) Inventors: Linda Einbond, Crestwood, NY (US); Stepehn Redenti, New York, NY (US)

(21) Appl. No.: 13/694,335

(22) Filed: Nov. 19, 2012

Related U.S. Application Data
(60) Provisional application No. 61/561,828, filed on Nov. 18, 2011.

(51) Int. Cl. A61K 31/7048; A61K 31/337; A61K 39/395; A61K 45/00; A61K 31/58

(52) U.S. Cl. CPC A61K 31/7048; A61K 45/00; A61K 31/58; A61K 39/3955; A61K 31/337
USPC 424/450; 424/451; 514/26; 514/173; 514/171; 424/133.1

(57) ABSTRACT

The present invention relates to pharmaceutical composition containing a physiologically effective dose of a nanoparticle triterpene glycoside or nanoparticle triterpene complex, wherein said complex is a liposome encapsulated compound, or exosome-encapsulated compound.

### CROSS-REFERENCE TO RELATED APPLICATION

This application claims the benefit of U.S. Provisional Application No. 61/561,828, filed Nov. 18, 2011, which is incorporated herein by reference in its entirety.

### FIELD OF THE INVENTION

This invention relates to novel nanoparticle complexes comprising triterpene glycosides and nanoparticles comprising triterpenes/aglycones, either alone or combined with chemopreventive or chemotherapy agents, useful to treat or prevent cancer, and other types of diseases.

### BACKGROUND OF THE INVENTION

Breast cancer is the second most common type of cancer worldwide. In 2012, it is estimated that approximately 290,170 women will be diagnosed with breast cancer. There will be 39,510 breast cancer deaths. The scientific and technical advances in breast cancer treatment have entailed a large cost. The treatment of breast cancer consumes a large part of the healthcare budget: (2005) 15-20% of all cancer costs and 1% of the total healthcare budget (Lamerato et al., 2006). The economic burden for the year 2001 was $15-20 billion (Campbell and Ramsey, 2009). The lifetime per patient costs of breast cancer range from $20,000 to $100,000.

Problems with currently existing treatments for breast cancer therapy are: (a) Toxicity: most anti-cancer drugs are often not selective for tumor cells vs. normal cells; (b) Bioavailability: The efficacy of drugs, as well as herbal extracts/components, is limited by bioavailability. The development of effective and safe treatments is thus of high priority (Lamerato et al., 2006).

An objective of the present invention is to develop an effective and well-tolerated combination strategy for primary and secondary breast cancer chemoprevention and therapy.

Over the past decade, the inventors have established a research program focused on the discovery of active principles from edible plants useful in the prevention and/or treatment of cancer.

There is not a standard of treatment for breast cancer patients. In addition to mastectomy and radiotherapy, treatments include administration of hormonal, cytotoxic or biologic therapies to improve the long-term outcome. Tamoxifen has been the standard of care in hormonal anti-estrogen therapy. Many new therapies are now being tested for their efficacy compared to tamoxifen.

Exosome Diagnostics' (New York, N.Y.) platform is related to research showing that exosomes released from cancer cells contain mutant RNA transcripts of key genes. Encapsula Nanosciences LLC (Nashville, Tenn.) prepares liposomes conjugated to molecules of interest. The platform is described in [www.encapsula.com; www.exosomedx.com] which is incorporated herein by reference.

The present invention encompasses a unified approach to treat cancer and other diseases with liposomes or exosomes from plants conjugated to the respective active components of those plants.

Targeted therapies attack gene changes in cells that cause cancer. The main classes are monoclonal antibodies and small molecules. Frost and Sullivan analyst Misty Hughes (Frost and Sullivan Healthcare Practice, Volume 6, Issue 3—April 2008] predicts that future treatment strategies will concentrate on drugs with defined therapeutic potential and minimal side effects. Based on the inventors' studies, actein, a triterpene glycoside, is a member of the small molecule group and satisfies these criteria.

The present invention comprehends liposomes combined with triterpene glycosides such as actein or triterpenes (Li et al, 2005). Nanoparticle triterpene glycosides increase the solubility, stability, bioavailability and safety of triterpene glycosides, such as actein. Actein is characterized by HPLC compared with standards. The present invention also comprehends exosomes prepared from black cohosh (rhizomes, fruits or other parts) or other edible plants (Planta Analytica, CN) to enrich for this component (Sun et al., 2011).

This approach can be applied to other combinations of herbal and/or chemotherapy agents, as well as nanoparticles prepared from plant or animal tissues that target specific tissues. This strategy is conceived to be useful to prevent and treat cancers and other diseases such as inflammatory diseases, HIV, lipid disorders, and osteoporosis.

Actein, similar to the synthetic triterpene CDDO (Liby et al., 2007) which is in clinical trials, targets multiple pathways. Breast cancer is a multifactor disease. Breast cancer and colorectal cancer have been associated with mutations in 189 genes (average of 11 per tumor). The optimal treatment for cancer, it can be reasoned, most likely requires an agent or combination of agents that can correctly target multiple pathways with minimal toxicity, either alone or combined with existing cytotoxic and hormonal therapies.

### SUMMARY OF THE INVENTION

The present invention overcomes the toxicity in association with most current chemotherapy drugs, and the bioavailability problem of black cohosh, by using nanoparticles with triterpene glycosides (such actein) and nanoparticles with triterpenes, alone or in combination with chemopreventive or chemotherapy agents. This could be quickly tested in the clinic and may have a dramatic impact on breast cancer incidence and survival.

In one aspect, this invention builds on the finding that the triterpene glycoside actein and related components identified from black cohosh or other edible plants, either alone or in combination with chemopreventive or chemotherapy agents, have significant non-toxic anti-cancer effects.

Since the efficacy of black cohosh/actein is limited by solubility, membrane-bound nanoparticles are used to significantly increase the solubility and stability in vitro and bioavailability in vivo.

Nanoparticle triterpene glycosides increase the solubility, stability, bioavailability and safety of triterpene glycosides. The following were prepared: 1) liposomes (prepared by Encapsula NanoSciences and according to Li et al., 2005); and 2) exosomes from black cohosh (rhizomes) (Sun et al., 2010). Actein is characterized by HPLC compared with standards.

As comprehended by this invention, this, approach can be applied to other combinations of herbal and/or chemotherapy agents, as well as nanoparticles prepared from plant or animal tissues that target specific tissues. This strategy is useful to prevent and treat breast cancer, as well as other cancers and other diseases such as inflammatory diseases, HIV, lipid disorders, and osteoporosis.

This invention comprehends nanoparticle triterpene glycosides (such as actein) or triterpenes (such as cimigenol), used either alone or in combination with chemopreventive/chemotherapy agents, to prevent and treat breast and other cancers. The problems with current cancer treatments concern toxicity and bioavailabilty. These problems are overcome using the triterpene glycoside or triterpene compound (such as actein) and the described nanoparticles. Also, the present invention is superior to the state of the art in that it provides a unified approach to treat cancer and other diseases with liposomes or exosomes from plants conjugated to their respective active components.

In one aspect, the present invention provides a pharmaceutical composition containing a physiologically effective dose of a nanoparticle triterpene glycoside or nanoparticle triterpene complex; wherein said complex is a liposome encapsulated compound, or exosome-encapsulated compound.

In another aspect, the present invention provides a pharmaceutical composition containing: (a) a physiologically effective dose of the composition; and (b) a physiologically effective dose of another chemotherapeutic agent.

Preferably, in these aspects, the triterpene glycoside compound is selected from the group consisting of actein, cimifugoside, cimigenol glycoside, cimiracemoside A, and 23-epi-26-deoxyactein. Also, preferably, the triterpene compound is 25-acetyl-7,8-didehydrocimigenol 3-O-β-D-xylopyranoside. Preferably, the triterpene compound is cimigenol.

Preferably, the complex is approximately 100 nm to approximately 400 nm in diameter.

Preferably, the liposome is composed of DOPE and DOPC.

In one aspect, the present invention provides a method of treating cancer comprising systemically administering a pharmaceutical composition containing a physiologically effective dose of a nanoparticle triterpene glycoside or nanoparticle triterpene complex; wherein said complex is a liposome encapsulated compound, or exosome-encapsulated compound.

In some embodiments, the method further comprises administering a chemotherapeutic agent. Preferably, the chemotherapeutic agent is selected from the group consisting of paclitaxel, doxorubicin, 5-FU, Herceptin, tamoxifen, sulindac sulfide, thapsigargin, an MEK inhibitor and combinations thereof.

In some embodiments, the cancer is an ER+ breast cancer or a HER+ breast cancer and the triterpene glycoside is actein. Preferably, the method further comprises administering paclitaxel and/or Herceptin wherein the cancer is a HER+ breast cancer.

In some embodiments, the cancer to be treated is a colon cancer.

In one aspect, the present invention provides a method of treating an inflammatory disorder comprising systemically administering a pharmaceutical composition containing a physiologically effective dose of a nanoparticle triterpene glycoside or nanoparticle triterpene complex; wherein said complex is a liposome encapsulated compound, or exosome-encapsulated compound.

### BRIEF DESCRIPTION OF THE DRAWINGS

FIG. 1 is a graph showing the growth inhibitory effects of Liposome-Actein (Lipo-Actein) compared to actein on MCF 7 human breast cancer cells. The agents appeared so active by microscopy that photos were taken and MTT was added at 22 h after treatment. The IC50 values would be higher if MTT would have been added at the usual time (96 h). Liposomes alone have been shown to not have a significant effect (Narayanan et al., 2009).

FIG. 2 are photographs showing effect of Lipo-Actein and actein on MCF7 cells by microscopy (22 h). (Nikon TI Eclipse; 40×). Note: Lipo-Actein is >4-fold more active than Actein on MCF7 cells.

FIG. 3 is a graph showing a mechanism of action of actein.

FIG. 4 shows action potentials after treating MCF7 cells (preloaded with the calcium sensitive dye Fura-2) with actein (100 nM or 100 μM). The Fura-2 dye binds to Ca²⁺ ions inside the cells. The different hatch markings represent the action potentials of different cells. Actein appears to open calcium channels and thus releasing calcium into the cytoplasm. The cells quickly recover and return to the baseline calcium concentration. (80-100 nM).

FIG. 5 shows microscope images of MCF7 cells (preloaded with the calcium sensitive dye Fura-2) after treating with actein (100 nM or 100 μM). Note: The color scale shows the increase in calcium concentration for concentrations of 100 nM and 100 μM. The 100 μM concentration has higher color intensity as there is a higher calcium influx throughout the cell. As color shifts to red there is an increase in calcium concentration.

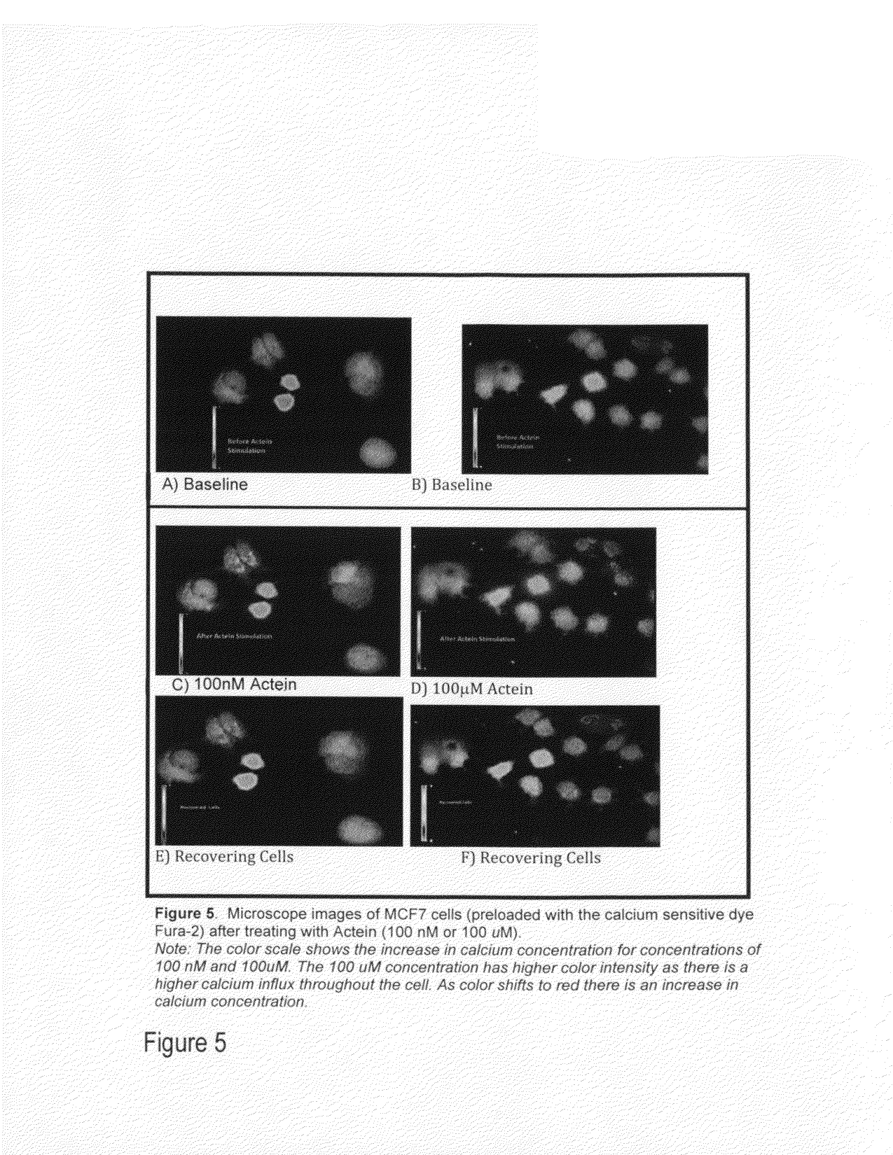

FIG. 6. Growth inhibitory activity of actein: A) alone; actein alone or combined with U0126 on: B) MCF7; C) HT29 cells; actein alone or combined with heparin on: D) MDA-MB-453 cells. Cells were exposed to increasing concentrations of agents for 96 h and the number of viable cells determined by the MTT assay.

FIG. 7. Action potentials after treating: A) MCF7 cells, preloaded with the calcium sensitive dye Fura-2; actein at 10 nM; 10 □M (different colors represent action potentials of different cells); B) images of the cells, by microscopy; C) MDA-MB-453 cells plus actein, in the presence or absence of heparin; D) HT29 cells plus actein or digitoxin.

FIG. 8. Synergistic combinations of thapsigargin and actein on MDA-MB-453 human breast cancer cells. We treated cells with all combinations of 3 concentrations of each of the agents tested and a solvent control. A) x-axis: thapsigargin; B) x-axis: actein; C) Combinations of tunicamycin and actein.

FIG. 9. Growth inhibitory effect of actein and liposome-actein (lipo-actein) on MCF7 cells: A) MTT assay: cells were treated with increasing concentrations of agents for 24 h. Since the components appeared highly active by microscopy, we added MTT at 24 h, instead of 96 h; B) microscope images; C) Schematic for the mode of action of actein; D) Structure of actein.

FIG. 10. Effect of actein on the NF-kB pathway. A) NF-kB activity; B) growth inhibitory effect of actein on the matched pair of cells 293T(null) and 293T(NF-kB). Cells were exposed to increasing concentrations of agents for 96 h and the number of viable cells determined by the MTT assay; C) Western blot analysis, as described in materials and Methods, of the effect of actein on the level of IKK□ protein. □-actin served as the loading control; D) Schematic of the effects of actein on the NF-□B pathway.

### DETAILED DESCRIPTION OF INVENTION

The present invention relates to pharmaceutical compositions for treating and preventing diseases and disorders, including, for example, cancer, and methods of treating such diseases and disorders.

Throughout this specification, quantities are defined by ranges, and by lower and upper boundaries of ranges. Each lower boundary can be combined with each upper boundary to define a range. The lower and upper boundaries should each be taken as a separate element.

A pharmaceutical composition of the present invention comprises a physiologically effective dose of an active compound encapsulated in a lysosome or encapsulated in an exosome, thereby forming a nanoparticle complex. In some embodiments, a pharmaceutical composition of the present invention consists essentially of (or consists of) the nanoparticle complex.

An active compound of the present invention comprises a triterpene glycoside and/or a triterpene. In some embodiments, an active compound consists essentially of (or consists of) a triterpene glycoside and/or a triterpene.

Preferred examples of a triterpene glycoside include actein, cimifugoside, cimigenol glycoside, cimiracemoside A, 23-epi-26-deoxyactein and 25-acetyl-7,8-didehydrocimigenol 3-O-β-D-xylopyranoside. A preferred example of a triterpene is cimigenol.

In the present invention, the nanoparticle complexes can range from approximately 20 nm to approximately 1000 nm in diameter, or from approximately 100 nm to approximately 500 nm in diameter, or from approximately 200 nm to approximately 400 nm in diameter.

In one embodiment, liposomes encapsulate active compounds to form nanoparticle complexes of the invention. The complex can be prepared by mixing a liposome solution and a solution containing an active compound. See Li et al., Cancer 2005 Sep. 15; 104(6):1322-31. for a method of preparation.

As is known in the art, liposomes are composed of lipid bilayers enclosing a hydrophilic core. These bilayers are formed from the lipophilic components. Negatively charged carboxylate groups stud the inner and outer surfaces of the liposomes. The diameter of a liposome complex increases as the amount of an active compound incorporated into the liposome increases.

The liposomes of the present invention are preferably fusogenic. For example a suitable liposome is composed of dioleoyl phosphatidylethanolamine (DOPE) and dioleoyl phosphatidylcholine (DOPC). Liposomes can also be prepared using other types of lipids, such as, for example, 1,2-Dimyristoyl-sn-glycero-3-phosphocholine (DMPC) and (DMPC/DMPG) (DMPG=1,2-dimyristoyl-sn-glycero-3-[phospho-rac-(1-glycerol)] [sodium salt]).

The ratio of total lipid to the active compound (weight/weight) can range from approximately 20:1 to approximately 2:1, or from approximately 15:1 nm to approximately 3:1, or from approximately 10:1 to approximately 4:1.

As a specific example, a preferred liposome complex is composed of DOPE:DOPC:Actein in 40:50:10 molar ratio in PBS buffer sized to 100 nm. The total lipid concentration is 23.2 mg/ml and the molar concentration of actein is 3.26 mM in liposome solution.

In some embodiments, chemopreventive and chemotherapeutic agents can also be added to the liposomes. For example, for combinations, a [lipo-active compound] and a [lipo-chemotherapeutic agent] can be mixed using a 3-way adjuvant mixer. See, for example, Narayanan et al., Int. J. Cancer, 2009, Jul. 1; 125(1):1-8.

In one embodiment, exosomes encapsulate active compounds to form nanoparticle complexes of the invention. Exosomes are 30-100 nm nanoparticles secreted by cells into the extracellular environment. Exosomes are prepared as known in the art. For example, see Sun et al., (Mol. Ther. 2010 September: 18(9):1606-14). In the present invention, exosomes are obtained from the species Actaea (e.g., black cohosh and Cimicifuga) and mixed with an active compound to form nanoparticle exosome complexes of the present invention. The diameter of an exosome complex increases as the amount of an active compound incorporated into the exosome increases. In some embodiments, the exosomes may contain the other constituents of black cohosh and Cimicifuga in addition to triterpene glycosides and triterpenes.

The nanoparticle complexes of the present invention significantly increase the solubility and stability in vitro and bioavailability in vivo of the active compounds.

In one aspect, present invention, provides methods of treating and preventing diseases and disorders. Preventing a diseases or disorder includes inhibiting and/or reducing the possibility of contracting a disease or disorder. The methods comprise systemic administration of the pharmaceutical compositions of the present invention to a mammal in need thereof. Systemic administration includes oral, intramuscular, intraperitoneal, subcutaneous and intravenous administration. Typically, the mammal is a human.

The pharmaceutical compositions of the present invention can be administered to treat and prevent cancer, for example, breast, prostate, oral, skin, colon and liver cancers. The compositions are particularly effective to treat breast and colon cancers. Treating includes inhibiting the growth of cancerous tumors.

Two major signaling pathways found in breast cancer cells are: the ER-mediated signaling pathway exemplified in the estrogen-dependent human breast cancer cell line MCF7 and the ER-negative Her2-mediated signaling pathway in the estrogen-independent human breast cancer cell line MDA-MB-453 which overexpresses Her2 (erb2, c-neu), a membrane-associated tyrosine kinase receptor. This invention comprehends targeting ER positive, as well as ER negative breast cancer, since actein preferentially targets Her2 overexpressing human breast cancer cells, which develop harder to treat and more aggressive tumors. The methods of the invention are particularly suited to treat ER+, HER+ and HER2+ breast cancer types.

In treating cancer, the pharmaceutical compositions of the present invention can synergistically be administered with chemopreventive and chemotherapeutic agents. Examples of such agents include paclitaxel, doxorubicin, 5-FU, Herceptin, tamoxifen, sulindac sulfide, thapsigargin and MEK inhibitor U0126. A particular synergistic result has been seen for the administration of actein in combination with paclitaxel and/or Herceptin to treat Her2 type breast cancer.

The chemopreventive agents and chemotherapeutic agents can be co-administered with the pharmaceutical compositions as a separate composition. Alternatively, the agents can coat the nanoparticle complexes of the invention.

The pharmaceutical compositions of the present invention target anti-inflammatory pathways. Thus, the compositions can be used to treat and prevent, and mediate the effects of inflammatory diseases, including, for example, cardiovascular and lipid disorders.

The pharmaceutical compositions of the present invention also have anti-HIV, statin, and osteoprotective effects. Methods of treating related disorders are included in the present invention.

Without wanting to be bound by a mechanism, it is believed that a mode of action of actein is as follows: 1) actein at low concentrations (100 nM) immediately opens calcium channels and releases calcium from cells; 2) actein encapsulated in liposomes is highly potent on human breast cancer cells (˜4-fold increase in activity).

The pharmaceutical compositions may be administered intermittently. For example, depending on the dose and the disease, the pharmaceutical compositions may be administered 1-6 times a day, preferably 1-4 times a day, as would be known to a skilled artisan.

Alternatively, the pharmaceutical compositions may be administered by sustained release. Sustained release administration is a method of drug delivery to achieve a certain level of the drug over a particular period of time. The level typically is measured by serum concentration.

For the pharmaceutical purposes described above, the nanoparticle complexes of the invention can be formulated per se in pharmaceutical preparations optionally with a suitable pharmaceutical carrier (vehicle) or excipient as understood by practitioners in the art. These preparations can be made according to conventional chemical methods.

For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums, glycols, buffers (e.g., PBS), and other pharmaceutically acceptable solvents.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the pharmaceutical compositions can be employed, and the pH of the solutions can be suitably adjusted and buffered. For intravenous use, the total concentration of the solute(s) can be controlled in order to render the preparation isotonic.

The pharmaceutical compositions of the present invention can further comprise one or more pharmaceutically acceptable additional ingredient(s) such as alum, stabilizers, buffers, coloring agents, flavoring agents, and the like.

Thus, while there have been described what are presently believed to be the preferred embodiments of the present invention, other and further embodiments, modifications, and improvements will be known to those skilled in the art, and it is intended to include all such further embodiments, modifications, and improvements and come within the true scope of the claims as set forth below.

### EXAMPLES

### Experiment 1

The preclinical studies are conducted in experimental systems of nonmalignant and malignant breast cells lines. The effects are compared of nanoparticle actein on the proliferation of nonmalignant mammary epithelial cells 184A1, obtained from Dr. Martha Stampfer (Lawrence Berkeley National Laboratory) and MCF1OF nonmalignant mammary epithelial cells with MCF7 (malignant, ER positive, Her2 low) and MDA-MB-453 (malignant, Her2 overexpressing, ER low). Also tested can be the effects of nanoparticle actein in the in vivo models of human ER-negative breast cancer.

### Making Nanoparticle Liposome Encapsulated Triterpene Glycosides

Fluorescent liposomes containing actein in the lipid bilayer were prepared as below: The liposomes produced are fusogenic and compose of DOPE:DOPC:Actein in 40:50:10 molar ratio in PBS buffer sized to 100 nm. The total lipid concentration will be 23.2 mg/ml and the molar concentration of actein will be 3.26 mM in liposome solution.

### Proliferation Assay of Liposomes Encapsulated Actein

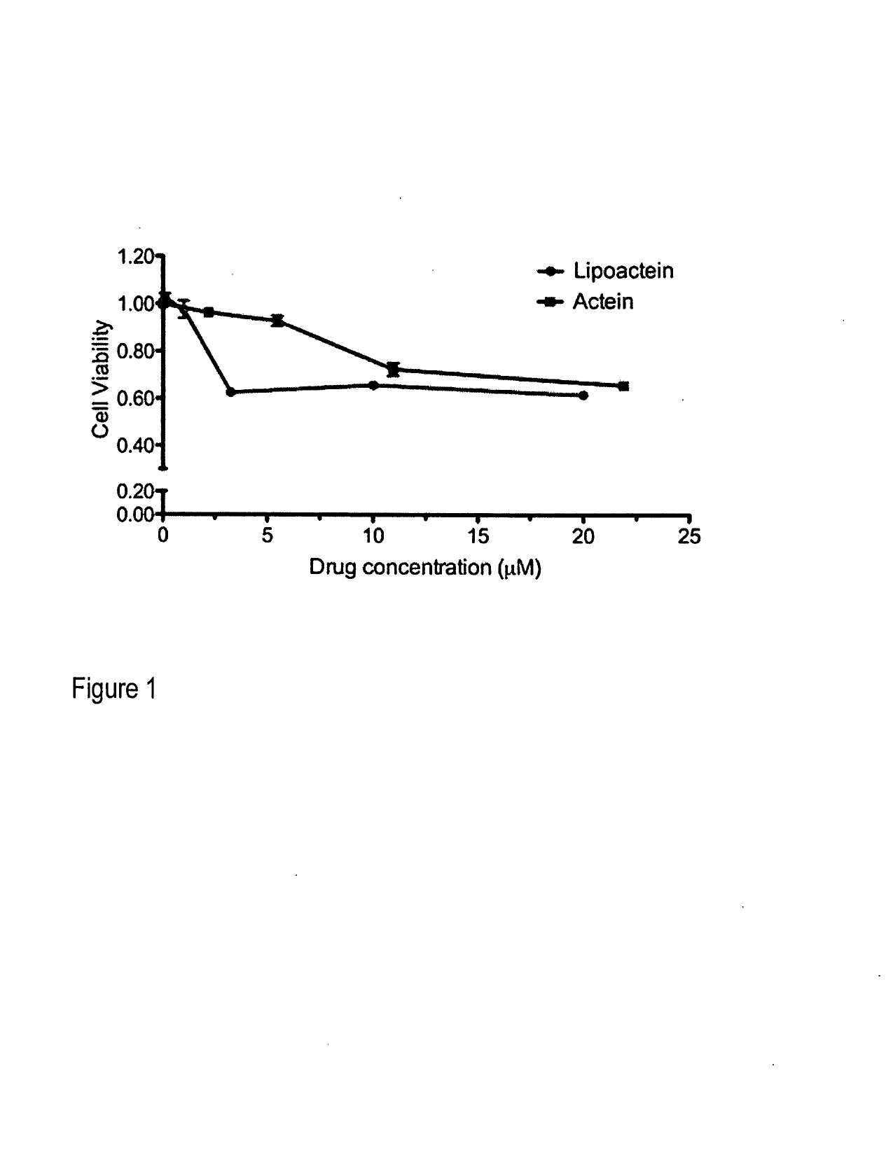

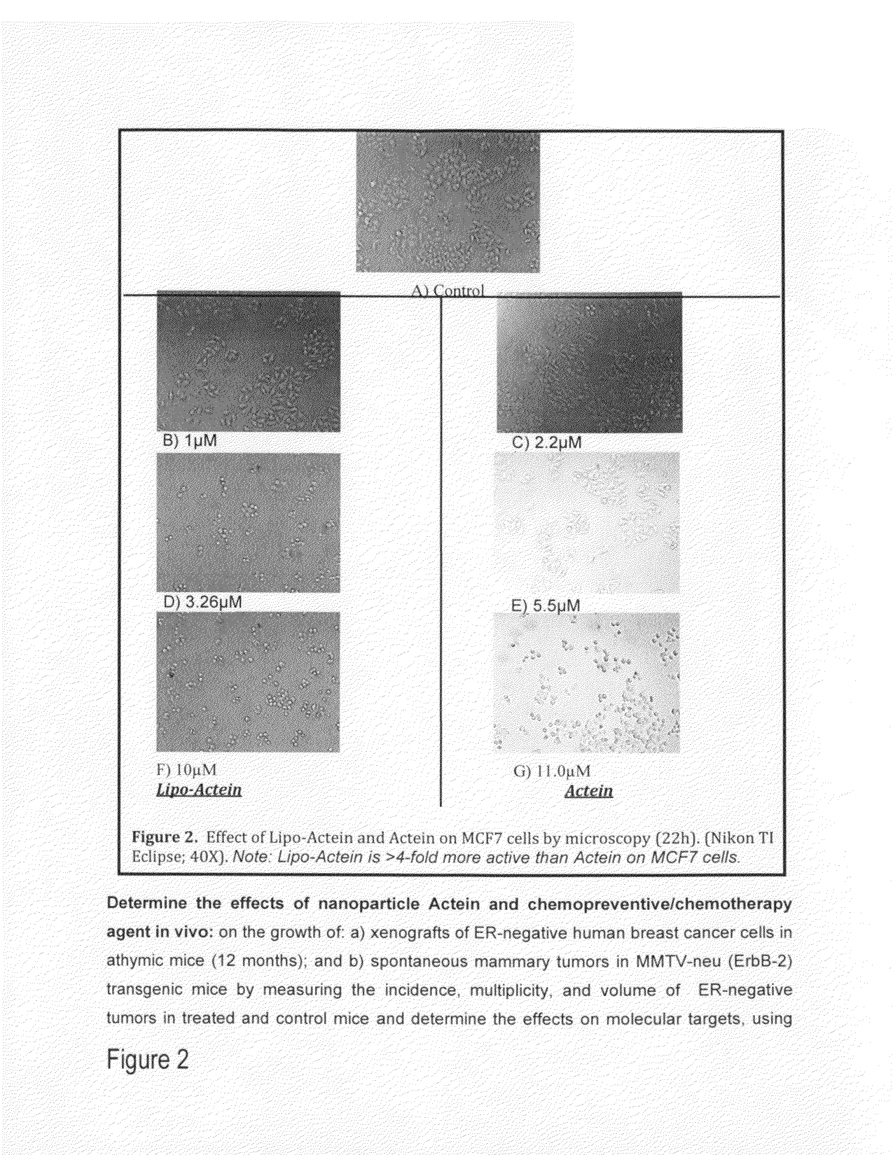

MCF7 ER positive Her2 low and MDA-MB-453 Her2 overexpressing ER-negative human breast cancer cells are treated with increasing concentrations of the liposome-actein complex and actein. The effects on the cell using microscopy at 22 h are examined and since the agents were highly active, photos were taken and an MTT assay performed (FIG. 1). IC50 values would be higher if measured at 96 h (FIGS. 1 and 2). FIG. 2 are photos of the cells. It is clear that in accord with the present invention, liposomes increase the activity of actein: liposome-actein is ˜4-fold more active than actein on MCF7 cells (actein alone at 11 μM is less active than lipo-actein at 3.4 μM).

### Materials

All solvents and reagents were reagent grade; H₂O was distilled and deionized. Naturex, Inc. (South Hackensack, N.J.) generously provided the black cohosh extract containing 27% triterpene glycosides, as previously described [3]. The extract of black cohosh enriched for triterpene glycosides (27%) contained 3.4% actein and 1.8% isoferulic acid; the most abundant components were cimicifugoside (5.0%) and cimigenol arabinoside (3.7%).

### Cell Cultures

MCF7 ER positive, Her2 low and MDA-MB-453 human breast cancer cells were obtained from the ATCC (Manassas, Va.). Cells were grown in Dulbecco's Modified Eagle's medium (DMEM) (Gibco BRL Life Technologies, Inc., Rockville, Md.) containing 10% (v/v) fetal bovine serum (FBS) (Gibco BRL) at 37° C., 5% CO₂.

### Assays for Growth Inhibition Using the Microtetrazolium (MTT) Assay

This method was used to determine the growth inhibitory effects of nanoparticle actein compared to actein in MCF7 human breast cancer cells (Einbond et al., 2007). For the MTT assay, cells were seeded at 1×10⁴ cells/well in 96-well plates and allowed to attach for 24 h. The medium was replaced with fresh medium containing nanoparticle actein in PBS or actein in DMSO (control: DMSO). The cells were treated for 96 h after which the cells were incubated with MTT reagents, according to the manufacturer's instructions (Roche Diagnostics), and the absorbance read at 575 and 650 nM. Control and treated cells were compared using the student's t-test (P<0.05). The IC50 values (i.e., the concentration that causes 50% inhibition) will be determined by linear regression analysis.

Method to Prepare Exosomes from Black Cohosh

Exosomes are membrane-bound nanopaticles (30-100 nm) which are released from cells, are present in blood and other bodily fluids and may act as an endocrine system. Exosomes have been shown to render curcumin more stable, soluble (5-fold) and bioavailable (Sun et al., 2010). Exosomes from fruits release nanoparticles that bind hydrophobic compounds such as curcumin. Intestine and immune cells in the intestine take up these particles, which should not induce negative side-effects since they are part of the diet and have a long history of safe use (James Graham Brown Cancer Center. Clinical Trial: NCT01294072). For more details, see Sun D et al., Mol. Ther. 2010 and Miller Donald, Study Investigating the Ability of Plant Exosomes to Deliver Curcumin to Normal and Colon Cancer Tissue; and James Graham Brown Cancer Center. Clinical Trial: NCT01294072.

Exosomes from black cohosh (rhizomes, fruits or other plant parts) or related plant species are also prepared (Sun et al., 2010) and tested for their effect on MCF7 ER positive Her2 low and MDA-MB-453 Her2 overexpressing ER-negative human breast cancer cells with increasing concentrations of the liposome-actein complex and actein.

The present invention comprehends that exosomes of black cohosh prepared as above can enrich and increase the bioavailability of actein and other triterpene glycosides produced by black cohosh.

This agent and method (exosomes) can also be used to prepare nanoparticle exosomes from other edible plants.

Compare the effects of nanoparticle actein in vitro: on the Proliferation of nonmalignant mammary epithelial cells and ER-negative human breast cancer cell lines, either alone or in combination with chemopreventive/chemotherapy agents.

Determine the absorption, distribution, metabolism, excretion, and toxicity of nanoparticle actein in vivo: either alone or combined with chemopreventive/chemotherapy agent using chemical microscopy and HPLC-MS analysis of mice serum, tissue, and urine samples.

Determine the effects of nanoparticle actein and chemopreventive/chemotherapy agent in vivo: on the growth of: a) xenografts of ER-negative human breast cancer cells in athymic mice (12 months); and b) spontaneous mammary tumors in MMTV-neu (ErbB-2) transgenic mice by measuring the incidence, multiplicity, and volume of ER-negative tumors in treated and control mice and determine the effects on molecular targets, using gene expression analysis, IHC, and Western Blot analysis of mammary tumors. (24 months).

### Mechanism of Action of Actein: Determine the Specific Signaling Pathways and Cellular Targets Involved in the Action of Actein

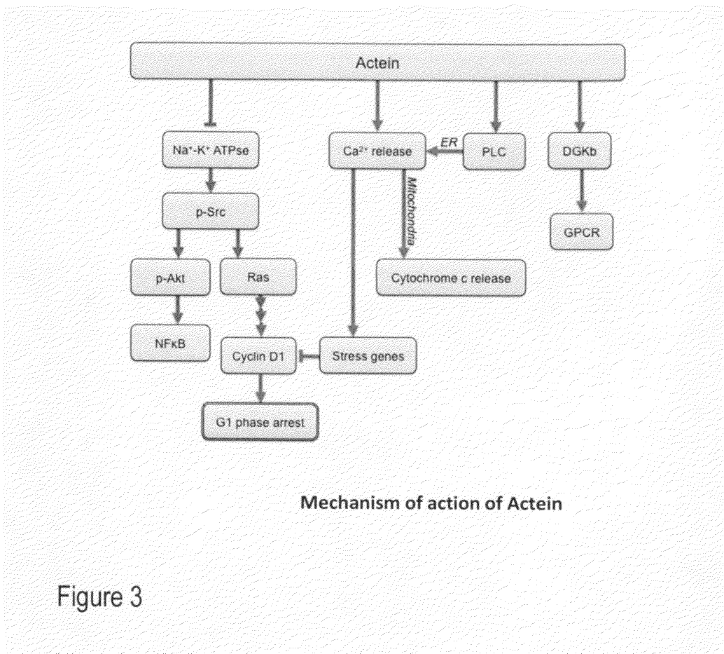

The mechanism of action of actein: (Schematic: FIG. 3) Gene expression, RT-PCR, Western blot and siRNA analysis indicated that Actein activates the expression of transcription factors that enhance apoptosis and represses the expression of survival and cell cycle genes. At early times (6 h), actein activated the integrated stress response (GRP78, BiP); DDIT4 (mTOR pathway) and lipid biosynthetic genes and inhibited the activity of the purified Na-K ATPase (Einbond et al., 2007; 2008). At later times (24 h), Actein reduced the level of NF-kB promoter activity, p-Erk, p-Akt as well as cyclin D1 protein levels (Einbond et al., 2008). In vivo, actein elicited stress and statin-associated responses in rat liver.

Although the exposure of cells to actein or the methanol extract of black cohosh can induce a complex array of cellular stress responses, the primary cellular molecules that actein or the methanol extract and related compounds target have not been identified (Einbond et al., 2007; 2007a). The putative targets may play a role in cellular processes involving calcium since actein altered the expression of several genes involved in calcium homeostasis, including GRP78, STC2, ADPN, JAG2, CALML5 and TRAM2 (Einbond et al., 2007).

Cardiac glycosides bind to the alpha subunit of the Na+/K+-ATPase; they act as potent and highly selective inhibitors of the active transport of Na+ and K+ across cell membranes (Goodman et al., 1966).

This leads to a small increase in intracellular Na+ and a large increase in intacellular Ca2+. The ability of cardiac glycosides to induce apoptosis may be related to the ability to generate an increase in Ca2+ ((McConkey et al., 2000). The inventors have found that actein also inhibits the activity of the purified Na+/K+-ATPase. Therefore, the effect of actein was tested on the intracellular level of calcium and a dramatic increase was found. FIG. 3 present a schematic of the mode of action of actein.

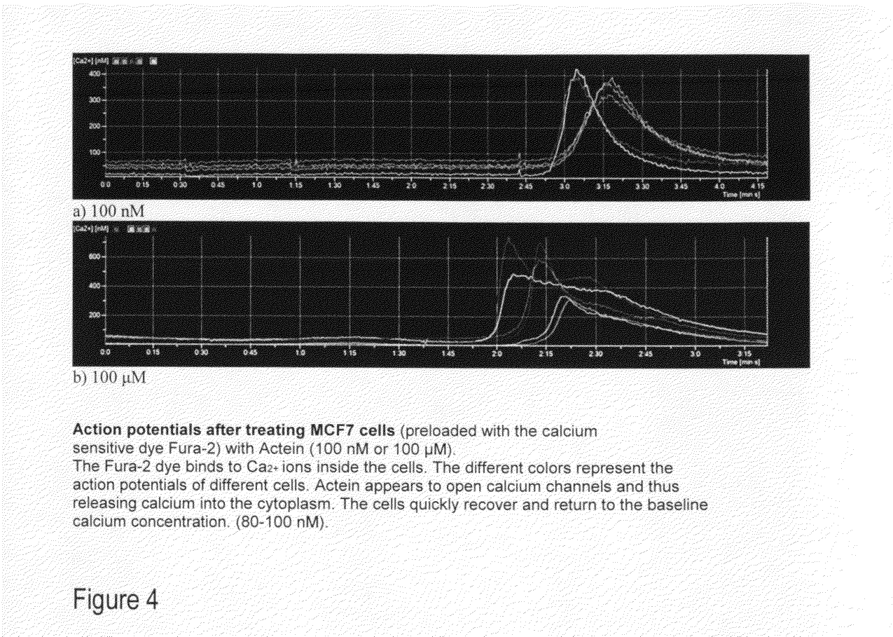

Mode of action of actein: 1) actein at low concentrations (100 nM or 100 mM) significantly increases intracellular calcium (ion concentration) levels in a dose-dependent manner (FIG. 4); at the 100 uM concentration there is a higher response of intracellular calcium. There was no significant change in baseline calcium levels when stimulated with the control DMSO.

To characterize the targets of nanoparticle actein affinity purification can be combined with mass spectroscopic proteomic analysis, comparing this with gene expression data to select proteins and then perform pulldown assays with tagged (biotinylated) actein (Yore et al., 2011).

### Experiment 2

The triterpene glycoside actein from the herb black cohosh, preferentially inhibits the growth of breast cancer cells and activates the ER stress response. Since actein is lipophilic, its action may be limited by bioavailability. Here the ability of nanoparticles to enhance the activity of actein to prevent and treat cancer was examined.

Methods: To reveal signaling pathways, human breast and colon cancer, as well as 293T(NK-kB), cells were treated with actein, alone or combined with specific inhibitors. Effects were measured using the MTT, luciferase, promoter and Western blot assays and histology. To assess effects on calcium release, cells were preloaded with the calcium sensitive dye Fura-2. To enhance bioavailability, actein was conjugated to nanoparticle liposomes.

Results: Actein strongly inhibits the growth of human breast and colon cancer cells. Actein induces a dose dependent release of calcium into the cytoplasm in human breast cancer cells. The IP3 antagonist heparin completely blocks this release and partially blocks the growth inhibitory effect of actein on human breast and colon cancer cells, indicating that the ER IP3R functions in the action. Consistent with this, actein synergizes with the ER mobilizer thapsigargin. Actein preferentially inhibits the growth of 293T(NF-κB) cells indicating that the NF-κB pathway is involved in its action. In addition, nanoparticle liposomes increase the growth inhibitory activity of actein. Conclusion: Actein appears to act through the ER IP3R, stress response and NF-κB pathways.

### Materials and Methods

### Materials

All solvents and reagents were reagent grade; H₂O was distilled and deionized. Agents: Actein (β-D-xylopyranoside, Planta Analytica, Danbury Conn.), thapsigargin and tunicamycin (Sigma, St. Louis, Mo.); U0126 (LC Laboratories, Woburn, Mass.) and Hygromycin (Invitrogen, Grand Island, N.Y.) were dissolved in dimethylsulfoxide (DMSO) (Sigma), while heparin (Sigma) was dissolved in deionized water, prior to addition to cell cultures.

### Cell Cultures

MDA-MB-453 Her2 and BT474 (ER+HER2+) overexpressing human breast and HT29 (p53 positive) human colon cancer cells were obtained from ATCC (MA). MCF7 human breast cancer cells were the generous gift of Dr. Moira Suane (CUNY, NY, N.Y.). MCF7, BT474 and MDA-MB-453 cells were grown in Dulbecco's Modified Eagle's medium (DMEM) (Gibco BRL Life Technologies, Inc., Rockville, Md.) containing 10% (v/v) fetal bovine serum (FBS) (Gibco BRL) and HT29 were maintained in McCoy's media plus 10% FBS, at 37° C., 5% CO₂. 293T(null) and 293T(NF-κB) luc stably transformed cells were obtained from Panomics and propagated in DMEM plus 10% FBS and P-S (Sigma).

### Proliferation Assay

MTT assay: the MTT (3-(4,5-dimethyl-2-thiazol)-2,5-diphenyl-2H tetrazolilum bromide) cell proliferation assay system (Roche Diagnostic, Mannheim, Germany) was used to determine the sensitivity of the various cell lines to agents. Cells were seeded at 1×10⁴ cells/well in 96-well plates and allowed to attach for 24 h. The medium was replaced with fresh medium containing DMSO or compound. The cells were treated for 96 or 120 h, after which they were incubated with MTT reagents and the absorbance read at 650 and 575 nm.

### Calculating the Combination Index

To determine the Combination Index (CI), cells were treated with all combinations of 3 (4 or 5) concentrations of each of the agents tested and a solvent control (Einbond et al., 2006). The results of the MTT assay were analyzed for possible synergistic effects using the median effect principle. Variable ratios of drugs were employed and mutually exclusive equations were assumed (Einbond et al 2006).

Microscopy: The effect of actein on human breast and colon cancer cells was examined by microscopy, using the Nikon TI Eclipse; 40×.

### NF-kB Luciferase Assay

A luciferase reporter construct is integrated in 293T cells and is regulated by 6 copies of the NFκB response element. The assay to monitor the activity of NFκB transcription factor was performed as described in the manufacturer's instructions (Panomics, Freemont, Calif.).

### Western Blot Analysis

Cells were treated for increasing times, in media containing serum, with approximately the IC₅₀ and twice the IC₅₀ concentration, measured at 48 h, of actein. Western blot analysis was performed as previously described (Einbond et al., 2004). The membrane was incubated with the primary antibody IKKβ (Santa Cruz Biotechnology; Santa Cruz, Calif.); β-actin was used as a loading control.

### Liposomes:

Liposomes-Actein: Actein encapsulated in liposomes, lipid: actein ratio 9:1 (DOPE:DOPC:actein=4:5:1) sized to 100 nM was prepared by Encapsula Nanosciences (Nashville, Tenn.).

### Gene Expression Analysis

Gene expression analysis was performed as previously described in vitro (Einbond et al., 2007)

### Statistical Analysis.

For cell growth, the data are expressed as mean+/−standard deviation. Control and treated cells were compared using the student's t-test, p<0.05 (Einbond et al., 2012).

AffyLimma analysis: To identify individual alterations in gene expression induced by treatment, an unbiased informatics analysis was performed using the AffyLimmaGUI package in the open-source Bioconductor suite, as previously described [Einbond et al., 2007.]

### Results

### Growth Inhibitory Activity of Actein

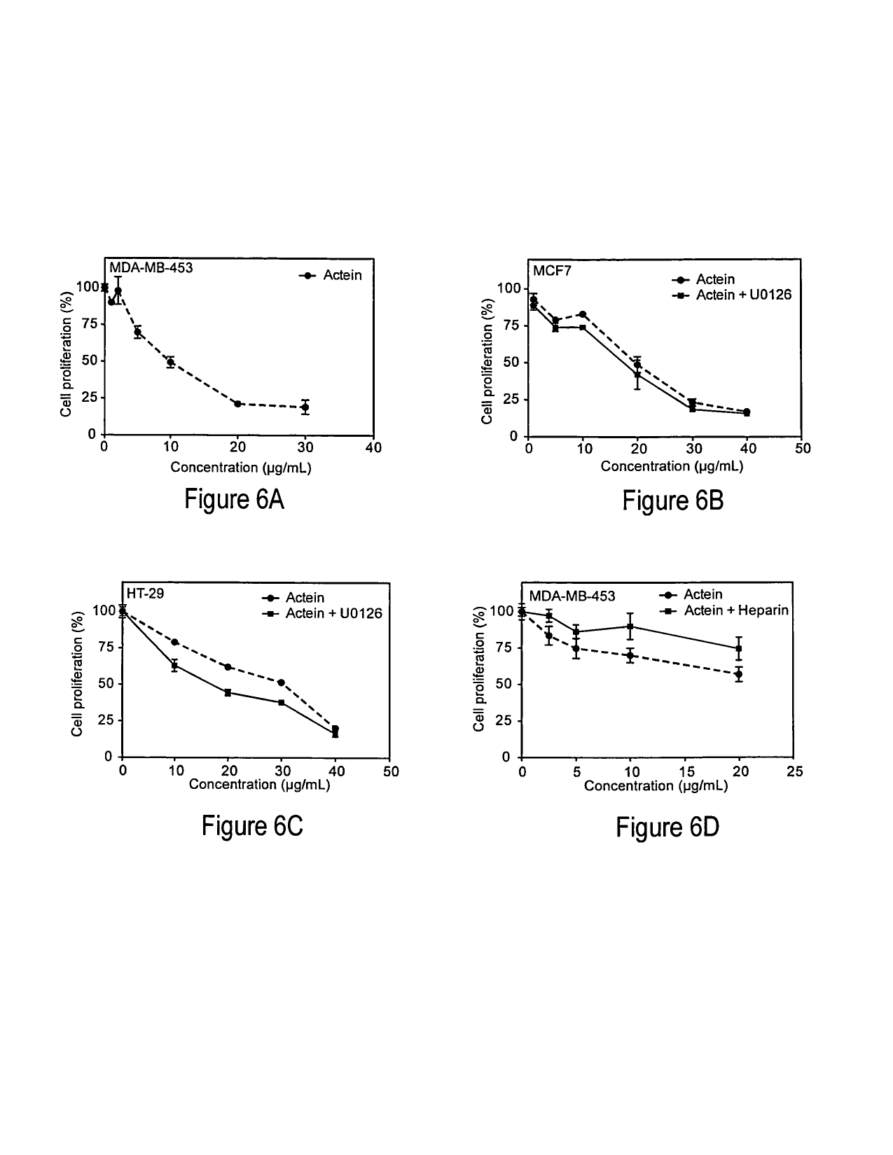

The growth inhibitory activity of actein on MCF7 (ER positive, Her2 low) and MDA-MB-453 (ER negative, Her2 overexpressing) breast and HT29 (p53 positive) colon cancer cells lines was examined. The IC₅₀ values, the concentration that caused 50% inhibition of cell growth, were approximately: MDA-MB-453 cells: 8 μg/ml (12 μM); MCF7 cells: 20 μg/ml (30 μM); (HT29): 24 μg/ml (35 μM) (FIG. 6A, B, C).

### Effects of the MEK Pathway Inhibitor U0126 on the Growth Inhibitory Effects of Actein

To reveal the signaling pathways of actein, the effect of actein alone or combined with the MEK inhibitor U0126 was tested. U0126 moderately enhanced the growth inhibitory effects of actein on MCF7 breast or HT29 colon cancer cells. The IC₅₀ values were: (MCF7) actein: 20 μg/ml; actein+U0126: 17μg/ml. (HT29) actein: 30 μg/ml; actein+U0126: 16.5 μg/ml. (FIG. 6B, C). For MCF7 cells, the percent viable cells decreased from 79.2% after treatment with actein (5 μg/ml) alone to 74.1% after treatment with actein plus U0126 (10 μM) (p<0.01). For HT29 cell, the percent viable cells decreased from 79.1% after treatment with actein (10 μg/ml) alone to 62.9% after treatment with actein plus U0126 (10 μM) (p<0.01).

### Effects of the IP3 Antagonist Heparin on the Growth Inhibitory Effects of Actein

To reveal the primary targets of actein, the effect of actein alone or combined with the competitive IP3 receptor antagonist heparin was tested. Heparin partially blocked the effects of actein on MDA-MB-453 breast (FIG. 6D) or HT29 colon cancer cells; for MDA-MB-453 cells the IC₅₀ values were: actein; 23 μg/ml; actein+heparin: 37 μg/ml; (FIG. 6D). At a dose of actein 2.5 μg/ml, the percent viable cells increased from 79.34% with actein alone to 91.6% with actein plus heparin (0.5 mg/ml), p<0.01.

### Effect of Actein on Calcium Release

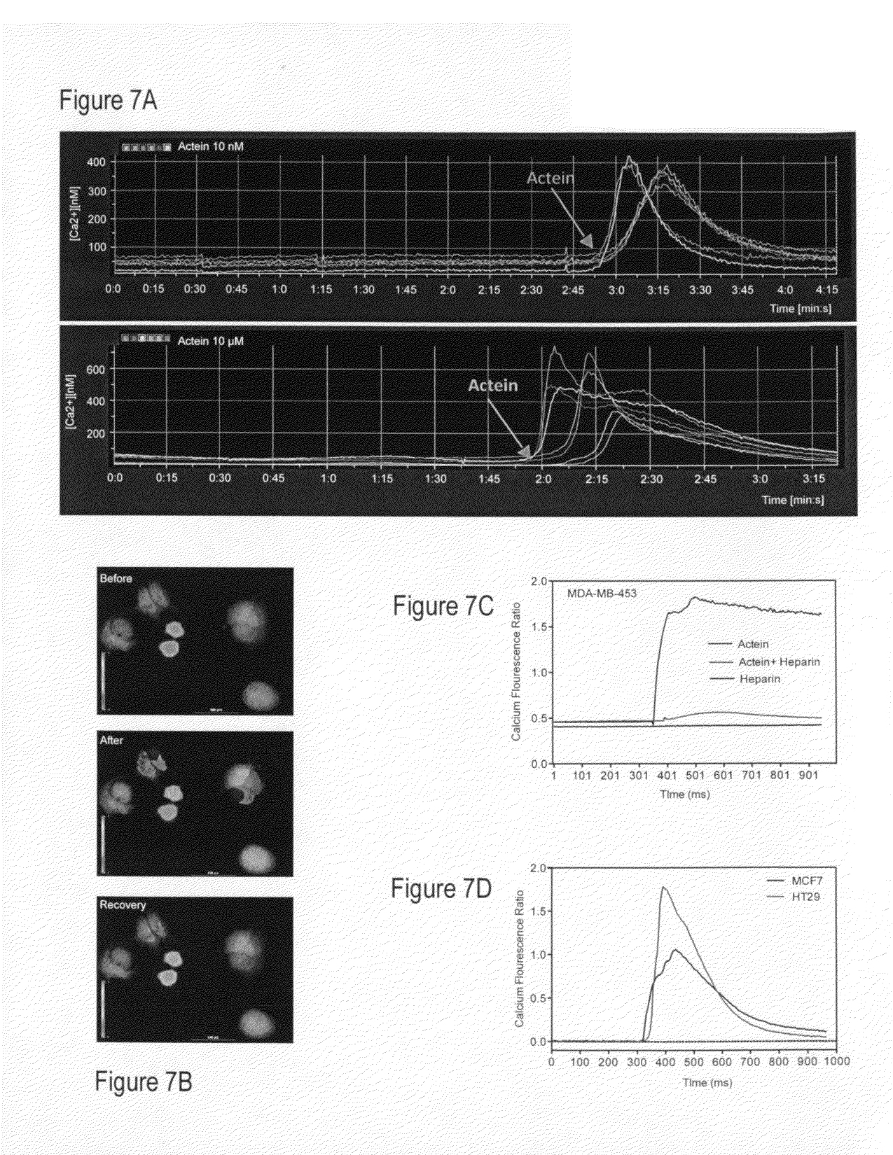

To elucidate the primary targets of actein, the effect of actein on calcium release was assessed. Cells were preloaded with the calcium sensitive dye Fura-2, which binds to calcium ions inside the cell. Actein induced an increase in action potential indicating calcium release, in two breast (MCF7, MDA-MB-453) and one colon cancer (HT29) cell line (FIG. 7A, B, C). The effect was dose dependent in that it was stronger at 10 μM than at 10 nM in MCF7 breast cancer cells (FIG. 7A, B). For MCF7 breast cancer cells, the effect was immediate and transient as the cells quickly recovered and returned to the baseline calcium concentration (80-100 nM). The increase in calcium level was more sustained at the higher dose of actein (10 μM)) versus 10 nM and it continued longer for MDA-MB-453 than for MCF7 breast cancer cells (FIG. 7C).

Effect of Actein, Alone or Combined with Heparin, on Calcium Release

Heparin completely blocked the effects of actein on calcium release in MDA-MB-453 breast cancer cells (FIG. 7C)

### Synergistic Combinations of Thapsigargin and Actein.

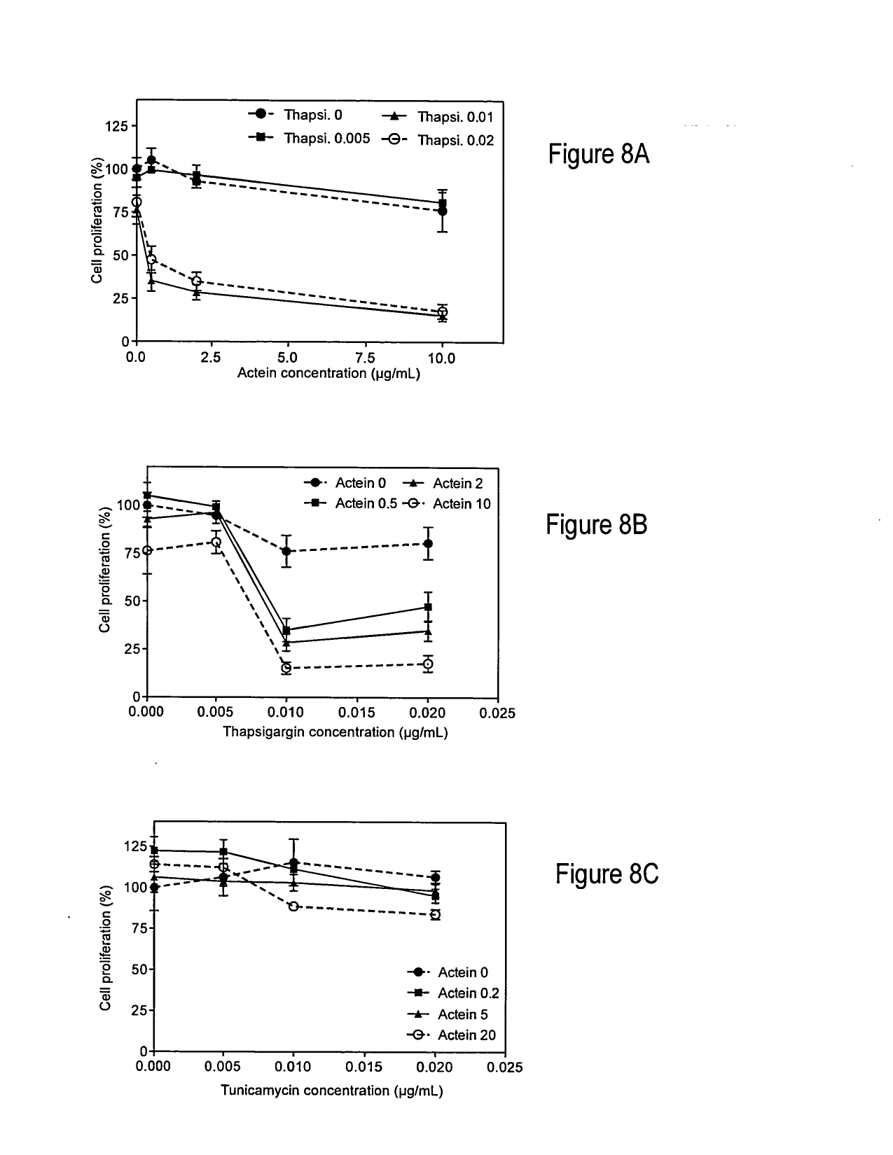

Next examined was the ability of actein to potentiate the effects of the ER mobilizer thapsigargin (SERCA, sarcoplasmic/Er Ca2+-ATPase inhibitor) and tunicamycin, the inhibitor of N-linked glycosylation of proteins, on ER negative MDA-MB-453 breast cancer cells. Actein enhanced the effects of thapsigargin (FIG. 8A, B), but not tunicamycin (FIG. 8C). The IC₅₀ values for actein and thapsigargin alone were 25 μg/ml μg/ml (μM) and 0.018 μg/ml μg/ml (μM), respectively. Then increasing concentrations of actein with increasing concentrations of thapsigargin were combined (FIG. 8A, B). At a dose of thapsigargin 0.01 μg/ml (μM), the percent viable cells decreased from 76.3% with thapsigargin alone to 35.2% with actein 0.5 μg/ml (μM), to 28.6% with actein 2 μg/ml (μM), to 15.3% with actein 10 μg/ml (μM) (p<0.01). The combination index for the combination of actein (0.5 μg/ml μg/ml) and thapsigargin (0.01 μg/ml) was approximately 0.58 indicating strong synergy (See Table 1). Thus actein enhanced the growth inhibitory effect of thapsigargin on MDA-MB-453 triple negative breast cancer cells.

TABLE 1
CI values for MDA-MB-453 cells treated with thapsigargin plus actein

| thapsigargin | actein (μg/mL) | | |
|---|---|---|---|
| (μg/mL) | 0.5 | 2.0 | 10.0 |
| 0.005 | 1.56 (−−) | 1.5 (−−) | 1.42 (−−) |
| 0.01 | 0.58 (+++) | 0.57 (+++) | 0.44 (+++) |
| 0.02 | 0.58 (+++) | 0.52 (+++) | 0.44 (+++) |

>1.3 (−−), antagonism;
1.1-1.3 (−), moderate antagonism;
0.9-1.1 (+−), additive effect;
0.8-0.9 (+), slight synergism;
0.6-0.8 (++), moderate synergism;
<0.6 (+++) synergism.
IC₅₀ values determined from the graphs in FIGS. 8A, B were used to obtain combination index values: CI = [IC₅₀ (thapsigargin + actein)/IC₅₀ (thapsigargin alone)] + [IC₅₀ (actein + thapsigargin)/IC₅₀ (actein alone).

### Effects of Actein on the NF-kB Pathway

The NF-kB family of transcription factors control the expression of genes which mediate immune responses, inflammation, cell survival and cancer (Hacker and Karin, 2006).

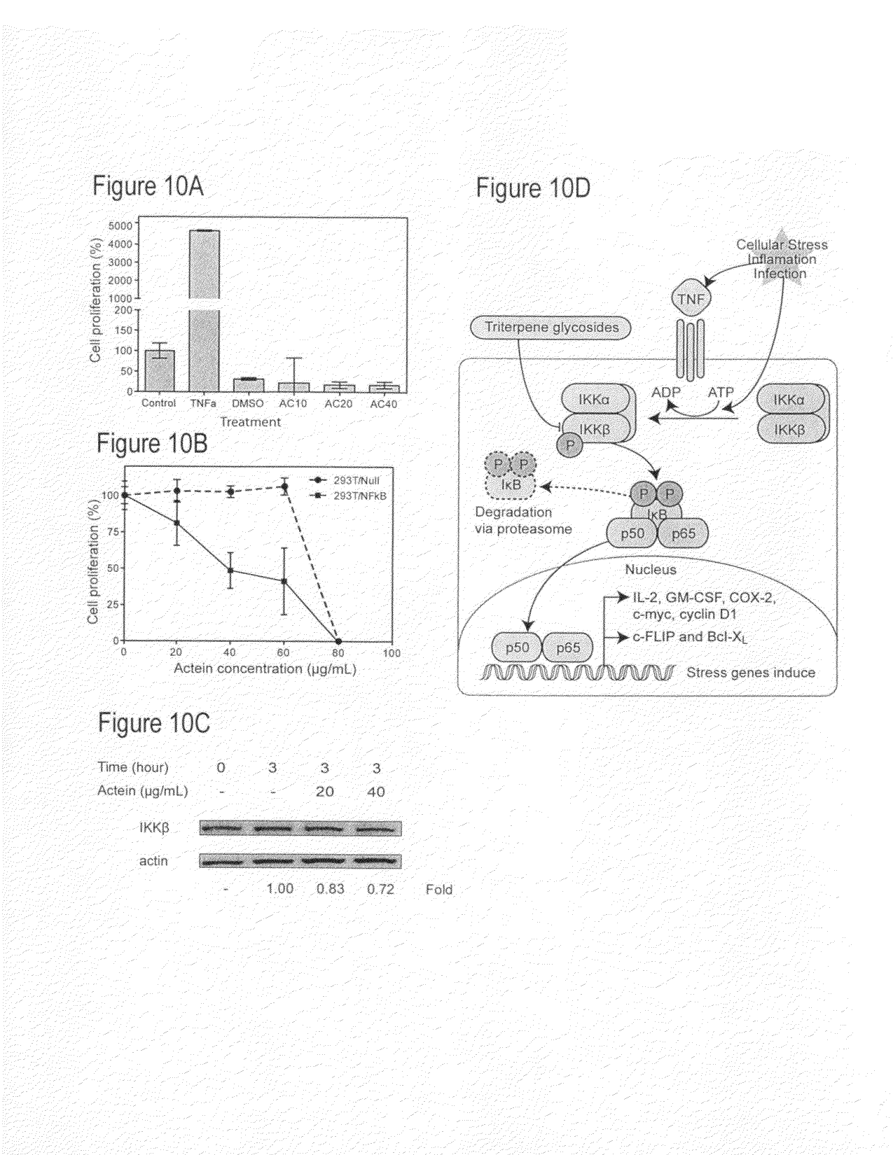

They are activated by such agents as cytokines, infectious agents and DNA double-strand breaks. To explore the effect of actein on the inflammatory response, the effect of actein on 293T cells stably transformed with the NF-κB plasmid were tested. It was found that actein did not increase cell proliferation; whereas, transforming growth factor alpha (TNFα) did (FIG. 10A).

The ability of actein to inhibit the growth of the parent 293T(null) and 293T(NF-κB) stably transformed cells was then compared. Actein was more effective on the transformed cells. The IC₅₀ values were: 293T(null): 75 μg/ml; 293T(NF-κB): 38 μg/ml (FIG. 10B). Since activation of NF-κB by, proinflammatory agents such as TNFα and lipopolysaccharide entail activation of IKKβ (and IKKγ) (Hacker and Karin, 2006), we examined the effect on IKKβ. Actein decreased the level of IKKβ protein in a dose dependent manner at 3 h (FIG. 10C) (Schematic FIG. 10D).

### Gene Expression Analysis of the Effect of Actein

Pathway express analysis indicated that actein (20 μg/ml) significantly modulated the expression of the inflammatory pathway, cytokine-cytokine interaction at 6 h in MDA-MB-453 breast cancer cells. Among the altered genes were: upregulated: EPOR; downregulated: CXCR4, TRAF5, IL17RB. Actein also significantly altered the expression of the MAPK signaling pathway including the gene (up) HSPA1A.

### Growth Inhibitory Effects of Actein and Lipo-Actein

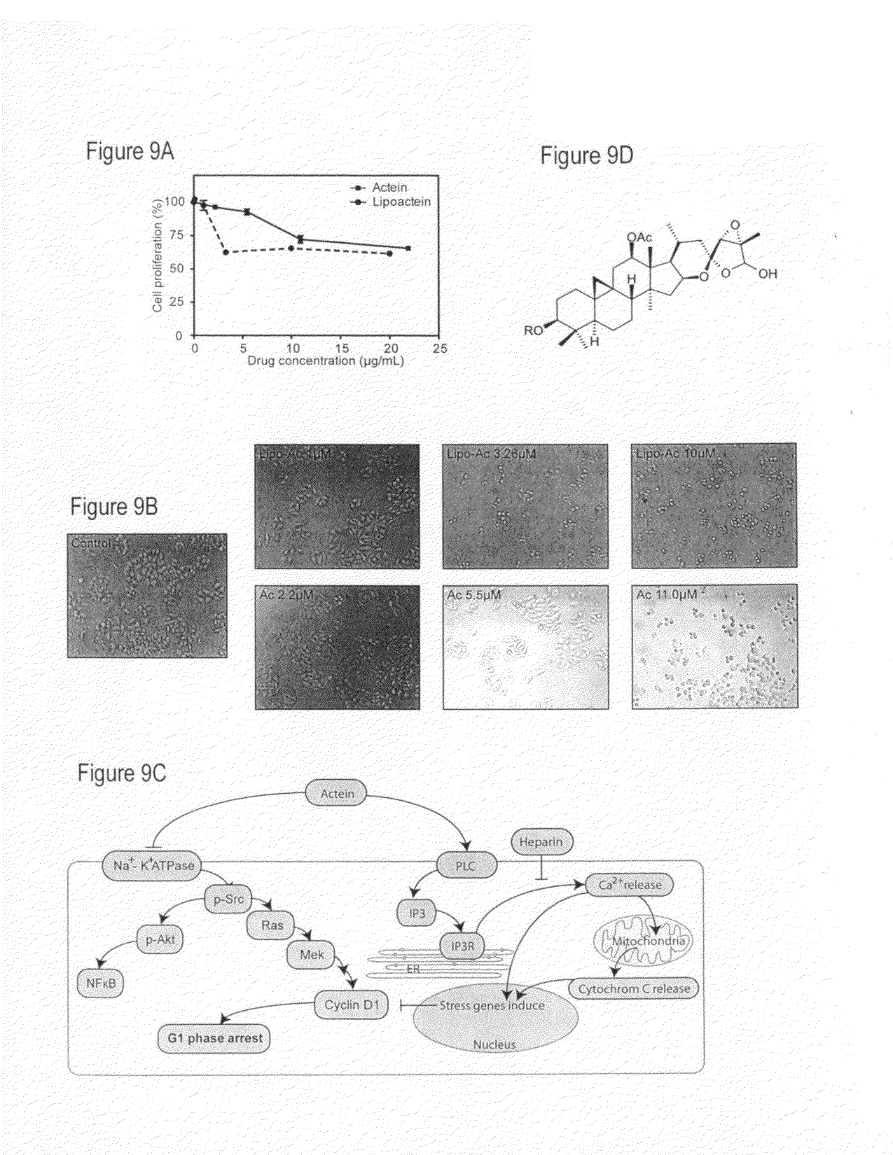

Actein encapsulated in liposomes were prepared and the growth inhibitory effect on MCF7 human breast cancer cells was assayed. Actein at 11 μM is less active than Lipo-Actein at 3.2 μM (FIG. 9A, B); thus Lipo-Actein is ˜4-fold more active than Actein on MCF7 cells.

### Findings

Calcium plays a role in multiple cellular processes including growth and tumorigenesis. Previous studies indicate that actein preferentially inhibits the growth of human breast cancer cells. The present inventors have shown for the first time that actein induces calcium release into the cytoplasm and that heparin completely blocks this release and partially blocks the growth inhibitory effect of actein on human breast and colon cancer cells. Consistent with this finding, actein synergizes with the ER mobilizer thapsigargin and the MEK inhibitor U0126, but not with tunicamycin, which inhibits N-linked glycosylation of proteins. Further, the present results indicate that actein preferentially inhibits the growth of NF-kB transformed cells versus null cells, indicating that actein targets the NF-kB pathway. In addition, the present inventors have shown that nanoparticle liposomes increase the growth inhibitory effects of actein.

Kumar et al. (2012) used specific inhibitors such as heparin to delineate the mode of action of D7. Their studies indicate that D7 induces a sustained release of calcium from ER stores by activating PC-PLC and oxidative stress. D7 appears to alter the plasma membrane thereby activating PLC which leads to the conversion of PIP4 to IP3 and diacylglycerol; IP3 binds to IP3 sensitive calcium channels in the ER (IP3R) leading to a release of calcium from the ER into the cytoplasm. This increase releases proapoptotic proteins into the cytoplasm which induce: 1) calpain-caspase12 apoptosis; and 2) mitochondrial pathways of apoptosis and cytochrome c release.

The instant results indicate that actein also (targets the ER IP3 and) induces the release of calcium. Kumar et al. (2012) found that D7 induced a sustained release of calcium into the cytoplasm, whereas actein induced a transient effect, which continued for longer duration at higher doses (10 μM versus 10 nM) and in MDA-MD-453 compared to MCF7 cells.

To reveal the molecular targets of actein, in particular, the effects on calcium release, the inventors treated HT29 colon cancer cells with actein alone or combined with the competitive IP3 antagonist heparin. Among its properties, heparin is not membrane-permeant, induces RyR, and uncouples G-protein coupled receptors from G proteins (Taylor and Tovey, 2010). Heparin partially blocked the growth inhibitory effect of actein on human breast and colon cancer cells. Thus, ER IP3R may be one of the molecular targets altered by actein. Since heparin completely blocked the release of calcium induced by actein, the ER IP3R appears to be required for this effect. Studies of others indicated that treatment with IP3 results in calcium binding to a stimulatory site (possibly in the IP3R) which opens calcium pores and prevents calcium binding to an inhibitory site (Taylor and Tovey, 2010); thus actein may alter the level of IP3. Since heparin blocked the increase in calcium, the ER appears to be the main source of calcium. The instant finding that actein synergizes with the ER mobilizer thapsigargin is consistent with these results (FIG. 9C).

Previous studies indicate that actein inhibits the activity of the Na/K ATPAse, a target of cardiac glycosides such as digitoxin. Actein's inhibition was less potent, but actein synergized with digitoxin on Na/K ATPase activity and breast cancer cell growth (Einbond et al., 2008). Further, actein induced a biphasic response on proteins downstream of the ATPase; at low doses or early times, actein activated the expression of NF-kB promoter activity, pAkt, pErk and cyclin D1 protein level, whereas at higher doses or later times, actein repressed these activities. (Einbond et al., 2008) (FIG. 9C). This is consistent with actein's biphasic response on the stress response. Black cohosh and actein induced two phases of the ISR—the survival or apoptotic phase depending on the dose and duration of treatment (Einbond et al., 2007a).

The biphasic effect of actein may relate to effects on calcium metabolism. Calcium regulates all IP3R in a biphasic pattern (Taylor and Tovey, 2010). The response of calcium binding proteins to transient and sustained signals can differ. Modest increases enhance responses to IP3 whereas higher doses inhibit the response (Taylor and Tovey, 2010).

Consistent with these findings, treatment with actein at 35.7 mg/kg for 6 h, downregulated the genes erythropoietin, CYP2C and ATP synthase in rat liver, suggesting that the primary effect of actein may be on hypoxia, the stress response and mitochondrial oxidative phosphorylation (Einbond et al., 2009).

To delineate the signaling pathways altered by actein, the effect of the MEK inhibitor U0126 was examined and it was found that it enhanced the growth inhibitory effect of actein. Mechanisms to explain the synergy include (Pesakhov et al., 2010): 1) one component may stabilize another; 2) mutual stabilization due to antioxidant properties; 3) different agents may alter different pathways which converge on one target, such as activation of caspases; or may alter different parts of one pathway such as the MEK pathway (pErk); 4) different agents may inhibit different phases of the cell cycle; and 5) one component may inhibit cellular mechanisms which mediate drug efflux.

The inventors also explored actein's effect on the NF-κB pathway. Actein was markedly more active on the transformed cell line 293T(NF-κB) than on the parent cell line indicating that the growth inhibitory effect of actein is due in part to an effect on the NF-κB pathway. Further actein reduced the expression of IKKα which will in turn decrease NF-κB activity.

Gene expression analysis confirmed anti-inflammatory effects in vitro and in vivo. Actein significantly altered the expression of the inflammatory pathway cytokine-cytokine interaction in MDA-MB-453 cells. In rat liver tissue; actein significantly altered the expression of the inflammatory pathway, acute phase response, and reduced the expression of the inflammatory gene CXCR4. In total, the instant results indicate that actein has the potential to modulate the inflammatory response and the NF-κB pathway.

The actein encapsulated nanoparticle liposome complexes of the present invention increased the activity of actein about 4-fold on MCF 7 human breast cancer cells. Thus nanoparticle liposome are useful to increase the activity—solubility, stability and bioavailability of actein in vivo.

In sum: Actein targets the IP3R, induces calcium release and modulates the NF-κB pathway inducing subsequent cell death.

### Experiment 3

Synergistic Effects of Actein in Combination with Paclitaxel or Herceptin:

The inventors tested actein alone, and in combination with chemotherapy agents, for growth inhibition of the ER− Her2 overexpressing breast cancer cell line MDA-MB-453. Actein exerted a synergistic effect when combined with paclitaxel or Herceptin. Moderate synergy (CI 2+) was seen with as little as 1 μg/mL of actein and 1 nM paclitaxel, and strong synergy (CI 3+) with 10 μg/mL actein and 1 nM paclitaxel. Since the MDA-MB-453 cells were relatively resistant to Herceptin (Yakes et al., 2002), the inventors tested the effect of actein in combination with Herceptin on the BT474 breast cancer cell line that expresses a high level of HER2. A 3+ synergistic effect was seen with 0.4 μg/mL actein plus 54 nM (8 μg/mL) Herceptin. Thus relatively low concentrations of actein can cause synergistic inhibition of human breast cancer cell proliferation when combined with the taxane paditaxel and the antibody to Her2 Herceptin.

We claim:

1. A pharmaceutical composition containing a physiologically effective dose of a nanoparticle triterpene glycoside or nanoparticle triterpene complex; wherein said complex is a liposome encapsulated compound, or exosome-encapsulated compound.

2. A pharmaceutical composition containing:
(a) a physiologically effective dose of the composition of claim 1; and
(b) a physiologically effective dose of another chemotherapeutic agent.

3. The composition of claim 1 wherein the triterpene glycoside compound is selected from the group consisting of actein, cimifugoside, cimigenol glycoside, cimiracemoside A, and 23-epi-26-deoxyactein.

4. The composition of claim 2 wherein the triterpene glycoside compound is selected from the group consisting of actein, cimifugoside, cimigenol glycoside, cimiracemoside A, and 23-epi-26-deoxyactein.

5. The composition of claim 1 wherein the triterpene compound is cimigenol.

6. The composition of claim 2 wherein the triterpene compound is cimigenol.

7. The composition of claim 1 wherein the triterpene compound is 25-acetyl-7,8-didehydrocimigenol 3-O-β-D-xylopyranoside.

8. The composition of claim 2 wherein the triterpene compound is 25-acetyl-7,8-didehydrocimigenol 3-O-β-D-xylopyranoside.

9. The composition of claim 1 wherein the complex is approximately 100 nm to approximately 400 nm in diameter.

10. The composition of claim 1 wherein the liposome is composed of DOPE and DOPC.

11. A method of treating cancer comprising systemically administering a pharmaceutical composition containing a physiologically effective dose of a nanoparticle triterpene glycoside or nanoparticle triterpene complex; wherein said complex is a liposome encapsulated compound, or exosome-encapsulated compound.

12. The method of claim 11 wherein the triterpene glycoside compound is selected from the group consisting of actein, cimifugoside, cimigenol glycoside, cimiracemoside A, 23-epi-26-deoxyactein and 25-acetyl-7,8-didehydrocimigenol 3-O-β-D-xylopyranoside.

13. The method of claim 11 wherein the triterpene compound is cimigenol.

14. The method of claim 11 further comprising administering a chemotherapeutic agent.

15. The method of claim 14 wherein the chemotherapeutic agent is selected from the group consisting of paclitaxel, doxorubicin, 5-FU, Herceptin, tamoxifen, and sulindac sulfide.

16. The method of claim 11 wherein the cancer is an ER+ breast cancer or a HER+ breast cancer and the triterpene glycoside is actein.

17. The method of claim 16 further comprising administering paclitaxel and/or Herceptin wherein the cancer is a HER+ breast cancer.

18. The method of claim 11 further comprising administering thapsigargin and/or an MEK inhibitor.

19. The method of claim 18 wherein the cancer is a colon cancer.

20. A method of treating an inflammatory disorder comprising systemically administering a pharmaceutical composition containing a physiologically effective dose of a nanoparticle triterpene glycoside or nanoparticle triterpene complex; wherein said complex is a liposome encapsulated compound, or exosome-encapsulated compound.
